(19) United States
(12) Patent Application Publication
(10) Pub. No.: US 2024/0076431 A1
(43) Pub. Date: Mar. 7, 2024

(54) COATING COMPOSITION COMPRISING A CARBAMATE-FUNCTIONAL POLY(ETHYLENE-ACRYLATE) COPOLYMER AND METHOD OF COATING SUBSTRATES

(71) Applicant: BASF Coatings GmbH, Müenster (DE)

(72) Inventors: Zhongliang ZHU, Troy, MI (US); Donald H. Campbell, Hartland, MI (US)

(21) Appl. No.: 18/259,722

(22) PCT Filed: Dec. 7, 2021
(86) PCT No.: PCT/EP2021/084530

§ 371 Date: Jun. 28, 2023

(30) Foreign Application Priority Data
Jan. 5, 2021 (EP) 21150250.5

(51) Int. Cl. C08F 220/20; B05D 7/00; C08F 4/18; C09D 133/06

(52) U.S. Cl. CPC C08F 220/20; B05D 7/532; C08F 4/18; C09D 133/066; C08F 2400/02; C08F 2810/50

(57) ABSTRACT

Disclosed herein are a method for producing materials having at least one carbamate functionality, carbamate-functional materials obtained from the process, coating compositions including said carbamate-functional materials, and a process for at least partially coating a substrate with the coating compositions. The method disclosed herein reduces unwanted transesterification which can occur as a side reaction by using a tertiary alkyl carbamate in combination with a tin catalyst and thus results in increased yields and purity of the carbamate-functional materials.

The present invention relates to a method for producing materials having at least one carbamate functionality, to carbamate-functional materials obtained from the inventive process, coating compositions comprising said carbamate-functional materials as well as a process for at least partially coating a substrate with said coating compositions. The inventive method reduces the unwanted transesterification which can occur as a side reaction by using a tertiary alkyl carbamate in combination with a tin catalyst and thus results in increased yields and purity of the carbamate-functional materials.

### STATE OF THE ART

Binder materials having carbamate groups have been used in thermosetting coating composition, for instance automotive clearcoat compositions. Such binder materials may be cured with relatively low viscosity aminoplast resins, which allow the coating formulations to have higher solids, to form cured coatings with excellent durability, including resistance to scratching, marring, and weathering degradation. Carbamate groups may be introduced into a binder material by reaction of a hydroxyl-functional material with an alkyl carbamate, for example methyl carbamate or butyl carbamate, through what is referred to as “transcarbamation” or “transcarbamoylation.”

The preparation of binder materials comprising carbamate functions is, for example, disclosed in U.S. Pat. No. 5,593,785 A and US 2004/0236031 A1. According to these documents, the carbamate-functional binders can be prepared by the reaction of hydroxyl groups of the binder with an alkyl carbamate, like methyl carbamate, in the presence of a catalyst, by decomposition of urea in the presence of hydroxyl groups or by reacting the hydroxyl groups of the binder with phosgene and subsequent treatment with ammonia.

Tin-based catalysts are the preferred catalysts for performing transcarbamation due to high yields. However, the use of alkyl carbamates, especially methyl carbamate, in combination with tin catalysts can lead to an undesired reaction of the alcohol formed as by-product with carbonate or ester functions present in the hydroxy-functional material. Said undesired reaction results in cleavage of the carbonate or ester functions and therefore in a significantly reduced yield and purity of carbamate-functional materials obtained from said transcarbamation.

Thus, there remains a need to prepare carbamate-functional materials using readily available carbamate sources in high yields and high purity. The method should be suitable for a large variety of hydroxy-functional materials and should be performed under mild reaction conditions. The resulting carbamate-functional materials should be suitable as binders in coating compositions, preferably without further purification steps.

### OBJECT

Accordingly, the object of the present invention is to provide a method for the production of carbamate-functional materials in high yields using readily available carbamate sources. The method should mitigate or avoid undesirable reactions with further functional groups of the hydroxy-functional material and should be conductible under mild conditions. The obtained carbamate-functional materials should have a purity which is sufficient to use them as binders in coating materials, preferably clear coating materials, without further purification steps. Coating layers produced from said coating materials should have a high scratch resistance, good acid resistance, and good weathering stability.

### TECHNICAL SOLUTION

The objects described above are achieved by the subject matter claimed in the claims and also by the preferred embodiments of that subject matter that are described in the description hereinafter.

A first subject of the present invention is therefore a method for preparing a carbamate-functional material, wherein at least one alkyl carbamate compound of general formula (I)

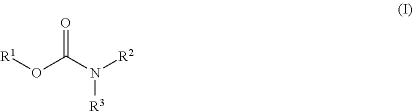

in which
R¹ is an organic residue bearing at least one tertiary carbon atom and optionally at least one aryl group and/or at least one heteroatom;
R², R³ are, independently from each other, selected from hydrogen or C₁ to C₁₀ alkyl groups,

is reacted with at least one compound having at least one hydroxy group in the presence of at least one tin containing catalyst.

The above-specified method is hereinafter also referred to as method of the invention and accordingly is a subject of the present invention. Preferred embodiments of the method of the invention are apparent from the description hereinafter and also from the dependent claims.

In light of the prior art it was surprising and unforeseeable for the skilled worker that the object on which the invention is based could be achieved by using a specific tertiary alkyl carbamate in combination with a tin containing catalyst. The use of the tertiary alkyl carbamate prevents undesired reaction of the formed alcohol with further functional groups present in the backbone of the hydroxy-functional compound and thus results in improved yields of the carbamate-functional materials. Due to the high purity, the resulting carbamate-functional materials can be used as binders in coating compositions without further purification steps. Moreover, the carbamate-functional materials are obtained under mild reaction conditions using readily available carbamate sources rendering the transcarbamation not only highly efficient but also very economical.

A further subject of the present invention is a carbamate-functional material prepared by the inventive method.

Moreover, another subject of the present invention is a coating composition comprising at least one inventive carbamate-functional material. The use of the carbamate-functional materials in coating compositions, particularly for a clearcoat layer or monocoat topcoat of automotive OEM finishes and refinishes, result in coating layers having a combination of high scratch resistance, good acid resistance, and good weathering stability.

A final subject of the present invention is a method of at least partially coating a substrate, comprising applying an inventive coating composition at least partly to the substrate and curing said applied coating composition.

### DETAILED DESCRIPTION

The measurement methods to be employed in the context of the present invention for determining certain characteristic variables can be found in the Examples section. Unless explicitly indicated otherwise, these measurement methods are to be employed for determining the respective characteristic variable. Where reference is made in the context of the present invention to an official standard without any indication of the official period of validity, the reference is implicitly to that version of the standard that is valid on the filing date, or, in the absence of any valid version at that point in time, to the last valid version.

All temperatures elucidated in the context of the present invention should be understood as the temperature of the room in which the substrate or the coated substrate is located. It does not mean, therefore, that the substrate itself is required to have the temperature in question. If room temperature is denoted in the following, this should be understood as a temperature ranging from 20 to 25° C.

### Inventive Transcarbamation Method

According to the inventive method, a carbamate-functional material is prepared by reacting a tertiary alkyl carbamate compound with a hydroxy-functional material in the presence of a tin containing catalyst. The carbamate-functional material thus represents a material having at least one carbamate group. The hydroxy-functional material may be a monomeric compound or a polymer and may have one or a plurality of hydroxyl groups.

“A,” “an,” “the,” “at least one,” and “one or more” are used interchangeably to indicate that at least one of the item is present; a plurality of such items may be present unless the context clearly indicates otherwise. All numerical values of parameters (e.g., of quantities or conditions) in this specification, including the appended claims, are to be understood to include all values and further divided ranges within the entire range. Each value within a range and the endpoints of a range are hereby all disclosed as separate embodiment. The terms “comprises,” “comprising,” “including,” and “having,” are inclusive and therefore specify the presence of stated items, but do not preclude the presence of other items. As used in this specification, using the term “or” includes any and all combinations of one or more of the listed items.

### Alkyl Carbamate Of General Formula (I):

The carbamate used in the inventive method is an alkyl carbamate compound of general formula (I)

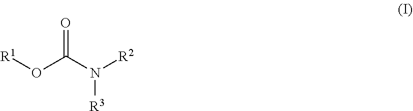

in which
R¹ is an organic residue bearing at least one tertiary carbon atom and optionally at least one aryl group and/or at least one heteroatom;
R², R³ are, independently from each other, selected from hydrogen or C₁ to C₁₀ alkyl groups.

Preferably, R¹ in general formula (I) is selected from aliphatic tertiary C₄ to C₁₂ alkyl residues, preferably aliphatic tertiary C₄ to C₁₀ alkyl residues, more preferably aliphatic tertiary C₄ to C₆ alkyl residues, very preferably aliphatic tertiary C₄ residues.

R² and R³ in general formula (I), independently from each other, are preferably selected from hydrogen.

With particular preference, the at least one alkyl carbamate compound of general formula (I) is tert-butyl carbamate. Use of tert-butyl carbamate in the inventive method prevents undesired side-reactions of the formed alcohol with further functional groups present in the backbone of the hydroxy-functional compound and thus results in improved yields of the carbamate-functional materials.

Compound having at Least One Hydroxy Group:

The compound having at least one hydroxy group which is reacted with the alkyl carbamate of general formula (I) described before is hereinafter also denoted as hydroxy-functional material.

The hydroxy-functional material may be a monomeric compound (i.e., a compound without a backbone composed of regularly repeating units), a resin, or a polymer and may have one or a plurality of hydroxyl groups. Oligomers are polymers having relatively few monomer units; generally, “oligomer” refers to polymers with only a few monomer units, perhaps up to ten; the term “polymers” is used to encompass oligomers as well as polymers with higher numbers of monomer units. Resins may be oligomers or compounds that do not have a backbone of regularly repeating monomer units, for example higher molecular weight compounds with one or more heteroatom-containing linking groups in addition to the hydroxyl group or groups. Resins may be dendrimers, hyperbranched, or “star” resins that are prepared from a polyfunctional core compound in one or more successive generations of branching reactants having one group reactive with the functionality of the core or of the latest generation to be added to the core and one or a plurality of groups available for reaction with the next generation of branching reactant.

In various embodiments, the hydroxyl-functional material may be a monomeric compound having exactly one hydroxyl group. Such monomeric compounds include aliphatic, cycloaliphatic, and aromatic mono-alcohols that may generally have from 1 to 160 carbon atoms, preferably 1 to 60 carbon atoms. The monomeric compounds may contain only hydroxyl groups or may contain heteroatoms such as O, S, Si, N, P in other groups such as ester groups, ether groups, amino groups, or unsaturated sites. Nonlimiting examples of suitable monomeric hydroxy compounds include straight and branched mono-alcohols having 1 to 60 carbon atoms and optionally including heteroatoms, for example butanol, decanol, 12-hydroxystearic acid, hydroxyalkyl (meth)acrylates including hydroxypropyl (meth)acrylate and hydroxyethyl (meth)acrylate, alkylene glycol monoalkyl ethers including propylene glycol monobutyl ether and monomethyl ether.

Moreover, the hydroxy-functional material may be a compound having—on average—more than one hydroxyl group. Preferably, the at least one compound having at least one hydroxy group has an average OH-functionality of 1.5 to 10, preferably 1.8 to 8, more preferably 1.8 to 6, very preferably 2 to 4.

Suitable compounds having at least one hydroxy group can be selected from polyols having 2 to 160 carbon atoms, polyester polyols, polyhydroxy polycarbonates, polyether polyols, polyurethane polyols, polyvinyl polymer polyols, polyhydroxy polyesteramides, polysiloxane polyols, polyhydroxy polythioethers, hyperbranched polyols and mixtures thereof.

Examples of polyols having 2 to 160 carbon atoms are 1,2-ethanediol, 1,3-propanediol, dimethylolpropane, 2-propyl-2-methyl-1,3-propanediol, 1,4-butanediol, 1,5-pentanediol, 1,2-hexanediol, 1,6-hexanediol, neopentyl glycol, 2-butyl-2-ethyl-,3-propanediol, 2,2-diethyl-1,3-propanediol, 2,2,4-trimethylpentane-1,3-diol, trimethylhexane-1,6-diol, 2-methyl-1,3-propanediol, 2-ethyl-1,3-hexanediol, ethyl-propyl-1,5-pentanediol, 2-methyl-2,4-pentanediol, 2,4,7,9-tetramethyl-5-decyn-4,7-diol, 2-butene-1,4-diol, pantothenol, dimethyltartrate, 3-[(hydroxymethyl)(dimethyl)silyl]-1-propanol, 2,2′-thiodiethanol, trimethylolethane, trimethylolpropane, trimethylolbutane 1,2,6-hexanetriol, glycerol, pentaerythritol and dipentaerythritol; cycloaliphatic diols such as cyclohexane dimethanol and cyclic formals of pentaerythritol such as, for instance, 1,3-dioxane-5,5-dimethanol; aromatic polyols, for instance 1,4-xylylene glycol and 1-phenyl-1,2-ethanediol, Bisphenol A, hydroquinone, and resorcinol; and monoethers and monoesters of polyols.

In certain embodiments, the polyol may include from 12 to 72 carbon atoms, preferably from 18 to 54 carbon atoms and more preferably from 36 to 54 carbon atoms, and at least two hydroxyl groups. The polyvalent radical bearing the hydroxyl groups may be substantially free of heteroatoms. The term “heteroatoms” refers to atoms other than carbon or hydrogen; the phrase “substantially free of” heteroatoms means that the polyvalent radical will generally have no more than two atoms, preferably no more than one atom, and more preferably no atoms other than carbon or hydrogen, e.g., atoms such as N, O, and Si. The polyvalent radical may be a structure or, preferably, a mixture of two or more saturated or unsaturated structures selected from the group consisting of noncyclic structures, aromatic ring-containing structures, cycloaliphatic structures. Saturated structures are preferred, especially where durability issues are of concern. Particularly advantageous mixtures are those having from 3 to 25 wt. % having an aliphatic structure, from 3 to 25 wt. % having an aromatic ring-containing structure, and 50 to 94 wt. % having a cycloaliphatic structure, preferably from 3 to 18 wt. % having an aliphatic structure, from 5 to 23 wt. % of reactive component having an aromatic-containing structure, and 55 to 85 wt. % of reactive component having a cycloaliphatic-containing structure. Most preferred mixtures of reactive component will comprise from 5 to 10 wt. % of reactive component having an aliphatic structure, from 10 to 20 wt. % of reactive component having an aromatic-containing structure, and 60 to 70 wt. % of reactive component having a cycloaliphatic-containing structure. Such polyol materials may be obtained by reduction of the carboxylic acid groups of dimerized, trimerized, tetramerized, or higher oligomer addition products of unsaturated fatty acids, particularly those with 12 to 18 carbon atoms. One particularly preferred polyol is a carbon diol with 36 carbon atoms. Such materials are commercially available from Croda International Plc. under the tradename Pripol™.

Further suitable hydroxyl-functional materials are hyperbranched polyol resins, monoalcohols and polyols such as the beta-hydroxy ester compounds resulting from the ring-opening of the oxirane ring of comprising at least one epoxide group by carboxylic acids as described in US 2015/0321998 A1.

Oligomeric and polymeric ethers which may be used include diethylene glycol, triethylene glycol, tetraethylene glycol, pentaethylene glycol, dipropylene glycol, tripropylene glycol, linear and branched polyethylene glycols, polypropylene glycols, and block copolymers of poly(ethylene oxide-co-propylene oxide). Other polymeric polyols may be obtained by reacting a polyol initiator, e.g., a diol such as 1,3-propanediol or ethylene or propylene glycol or a polyol such as trimethylolpropane or pentaerythritol, with a lactone or alkylene oxide chain-extension reagent. Similar polyester polyols may be obtained by reacting polyol initiator molecules with hydroxy acids, such as 12-hydroxystearic acid. In other embodiments, a polyol initiator compound may be reacted with an oxirane-containing compound to produce the respective polyether diol.

Polyurethanes having hydroxyl functional groups are also well known in the art. Examples of suitable polyurethane polyols include polyester-polyurethanes, polyether-polyurethanes, and polycarbonate-polyurethanes, including, without limitation, polyurethanes polymerized using as polymeric diol reactants polyethers and polyesters including polycaprolactone polyesters or polycarbonate diols. These polymeric diol-based polyurethanes are prepared by reaction of the polymeric diol (polyester diol, polyether diol, polycaprolactone diol, polytetrahydrofuran diol, or polycarbonate diol), one or more polyisocyanates, and, optionally, one or more chain extension compounds. Chain extension compounds, as the term is being used, are compounds having two or more functional groups, preferably two functional groups, reactive with isocyanate groups, such as the diols, amino alcohols, and diamines. Preferably the polymeric diol-based polyurethane is substantially linear (i.e., substantially all of the reactants are difunctional).

Polyvinyl polyols, such as acrylic (polyacrylate) polyol polymers that may be used as the hydroxy-functional material may be prepared by polymerizing one or more hydroxyl-functional, ethylenically unsaturated monomers with one or more other ethylenically unsaturated monomers. Suitable monomers are, for example, described in US 2015/0321998 A1.

A polysiloxane polyol may be made by hydrosilylating a polysiloxane containing silicon hydrides with an alkyenyl polyoxyalkylene alcohol containing two or three terminal primary hydroxyl groups, for example allylic polyoxyalkylene alcohols such as trimethylolpropane monoallyl ether and pentaerythritol monoallyl ether.

With particular preference, the at least one hydroxy-functional material is selected from polyester polyols and/or polyhydroxy polycarbonates.

Such polyester polyols can be prepared by reacting: (a) polycarboxylic acids or their esterifiable derivatives, together if desired with monocarboxylic acids, (b) polyols, together if desired with monools, and (c) if desired, other modifying components. Nonlimiting examples of polycarboxylic acids and their esterifiable derivatives include phthalic acid, isophthalic acid, terephthalic acid, halophthalic acids such as tetrachloro-or tetrabromophthalic acid, adipic acid, glutaric acid, azelaic acid, sebacic acid, fumaric acid, maleic acid, trimellitic acid, pyromellitic acid, tetrahydrophthalic acid, hexahydrophthalic acid, 1,2-cyclohexanedicarboxlic acid, 1,3-cyclohexane-discarboxlic acid, 1,4-cyclohexane-dicarboxlic acid, 4-methylhexahydrophthalic acid, endomethylenetetrahydropthalic acid, tricyclodecane-dicarboxlic acid, endoethylenehexahydropthalic acid, camphoric acid, cyclohexanetetracarboxlic acid, and cyclobutanetetracarboxylic acid. The cycloaliphatic polycarboxylic acids may be employed either in their cis- or in their trans-form or as a mixture of the two forms. Esterifiable derivatives of these polycarboxylic acids include their single or multiple esters with aliphatic alcohols having 1 to 4 carbon atoms or hydroxy alcohols having up to 4 carbon atoms, preferably the methyl and ethyl ester, as well as the anhydrides of these polycarboxylic acids, where they exist. Nonlimiting examples of suitable monocarboxylic acids that can be used together with the polycarboxylic acids include benzoic acid, tert-butylbenzoic acid, lauric acid, isonoanoic acid and fatty acids of naturally occurring oils. Nonlimiting examples of suitable polyols include any of those already mentioned above, such as ethylene glycol, butylene glycol, neopentyl glycol, propanediols, butanediols, hexanediols, diethylene glycol, cyclohexanediol, cyclohexanedimethanol, trimethylpentanediol, ethylbutylpropanediol ditrimethylolpropane, trimethylolethane, trimethylolpropane, glycerol, pentaerythritol, dipentaerythritol, tris-hydroxyethyl isocyanate, polyethylene glycol, polypropylene glycol. Nonlimiting examples of monoalcohols that may be used together with the polyols include butanol, octanol, lauryl alcohol, and ethoxylated and propoxylated phenols. Nonlimiting examples of suitable modifying components include compounds which contain a group which is reactive with respect to the functional groups of the polyester, including polyisocyanates and/or diepoxide compounds, and also if desired, monoisocyanates and/or monoepoxide compounds. The polyester polymerization may be carried out by known standard methods. This reaction is conventionally carried out at temperatures of between 180 and 280° C., in the presence if desired of an appropriate esterification catalyst. Typical catalysts for the esterification polymerization are protonic acids, Lewis acids, titanium alkoxides, and dialkyltin oxides, for example lithium octanoate, dibutyltin oxide, dibutyltin dilaurate, para-toluenesulfonic acid under reflux with small quantities of a suitable solvent as entraining agent such as an aromatic hydrocarbon, for example xylene, or a (cyclo)aliphatic hydrocarbon, for example cyclohexane.

Nonlimiting examples of suitable polyhydroxy polycarbonates that might be used include those prepared by the reaction of polyols with dialkyl carbonates (such as diethyl carbonate), diphenyl carbonate, or dioxolanones (such as cyclic carbonates having five- and six-member rings) in the presence of catalysts like alkali metal, tin catalysts, or titanium compounds. Useful polyols include, without limitation, any of those already mentioned. Aromatic polycarbonates are usually prepared from reaction of bisphenols, e.g., bisphenol A, with phosgene or diphenyl carbonate.

With particular preference, the polyester polyol is a reaction product of epsilon caprolactone and pentaerythritol.

Particularly preferred polyester polyols have a weight average molecular weight M_{w} of 500 to 2,000 g/mol, preferably 600 to 1,500 g/mol, very preferably 900 to 1,200 g/mol, as determined according to gel permeation chromatography using polystyrene as internal standard.

Moreover, particularly preferred polyester polyols have a hydroxyl number of 150 to 400 mg KOH/g solids, preferably 180 to 350 mg KOH/g solids, more preferably 200 to 300 mg KOH/g solids, very preferably 215 to 235 mg KOH/g solids, as determined according to R.-P. Krüger, R. Gnauck and R. Algeier, Plaste and Kautschuk, 20, 274 (1982).

With particular preference, the polyhydroxy polycarbonate is prepared by reacting carbon acid compounds, preferably diphenyl carbonate and/or dimethyl carbonate and/or phosgene, with diols, preferably 1,5-pentane diol and/or 1,6-hexane diol.

Particularly preferred polyhydroxy polycarbonates have a weight average molecular weight M_{w} of 500 to 3,000 g/mol, preferably 700 to 2,500 g/mol, very preferably 950 to 2,000 g/mol, as determined according to gel permeation chromatography using polystyrene as internal standard.

Moreover, particularly preferred polyhydroxy polycarbonates have a hydroxyl number of 30 to 300 mg KOH/g solids, preferably 35 to 200 mg KOH/g solids, more preferably 40 to 150 mg KOH/g solids, very preferably 50 to 65 mg KOH/g solids or 100 to 120 mg KOH/g solids, as determined according to R.-P. Krüger, R. Gnauck and R. Algeier, Plaste and Kautschuk, 20, 274 (1982).

It is preferred if a specific molar ratio of the at least one alkyl carbamate compound of formula (I) and the at least one hydroxy-functional material is used in the inventive method. Preferred molar ratios of the at least one alkyl carbamate compound of general formula (I), preferably tert-butyl carbamate, to the at least one compound having at least one hydroxy group, preferably polyester polyol and/or polyhydroxy polycarbonate, are 1.5 to 5, more preferably 1.5 to 3, very preferably 1.5 to 2. Use of the aforementioned molar ratios allows a high degree of conversion of the hydroxyl groups of the hydroxy-functional material to carbamate groups.

### Tin Containing Catalyst

The reaction between the hydroxy-functional material and the alkyl carbamate of general formula (I) is performed in the presence of at least one tin containing catalyst.

Suitable tin containing catalysts are selected from dialkyltin alkanoates, dialkyl tin oxides and mixtures thereof. With particular preference, dibutyltin oxide is used as tin containing catalyst. The use of dibutyltin oxide in combination with the alkyl carbamate of general formula (I) allows to obtain the carbamate-functional material in high yields and purity and prevents undesired side reactions of the formed alcohol with the hydroxy-functional material.

Preferably, the at least one tin containing catalyst, preferably dibutyltin oxide, is used in a total amount of 0.01 to 1 wt. %, more preferably 0.02 to 0.5 wt. %, even more preferably 0.03 to 0.3 wt. %, very preferably 0.05 to 0.2 wt. %, based in each case on the total amount of the at least one alkyl carbamate compound of general formula (I) and the at least one compound having at least one hydroxy group.

### Further Reaction Conditions

The transcarbamation is preferably carried out in the absence of oxygen, for example under a nitrogen atmosphere. The nitrogen blanket may be removed as the temperature begins to approach reflux as long as the nitrogen is resumed once reflex is lost. The reaction vessel should be equipped with suitable stirring, heating and cooling equipment as well as with a reflux condenser which condenses volatile constituents, for example solvent and alcohol by-product from the transcarbamation reaction. A trap or some other device may also be included for removing the alcohol by-product. The transcarbamation reaction may use toluene to aid in removing the by-product and may be carried out ata temperature in the range of from 110° C. to 140° C. An optimum temperature for the transcarbamation reaction may be determined by straightforward experimentation, and depends on factors, as should be expected, such as temperature, reactant concentrations, and solubility in the particular solvent system. Mineral acids such as phosphoric acid should be avoided. As may be expected, a certain minimum temperature may need to be reached for the reaction to progress at a desired rate.

In principle, the transcarbamation reaction can be carried out in any organic solvent which is inert towards the hydroxy-functional material, the alkyl carbamate of general formula (I) and the produced carbamate-functional material. Suitable organic solvents are, for example, selected from aromatic hydrocarbons, preferably toluene, xylene, mesitylene, 2-, 3-, or 4-ethyltoluene; naphthas; aliphatic and cycloaliphatic hydrocarbons, preferably white spirits, cyclohexane, mineral turpentine, tetralin and decalin; ketones, and mixtures thereof, preferably toluene.

The progress of the transcarbamation reaction may be carried out by monitoring hydroxyl number of the hydroxyl-functional material or by monitoring the amount of by-product alcohol (e.g., methanol methyl carbamate) collected. Since the use of the specific alkyl carbamate of general formula (I) in combination with the tin containing catalyst is specific to the transcarbamation reaction, the amount of by-product alcohol (e.g., tert-butanol when tert-butyl carbamate is used) matches the amount expected from titration of hydroxyl number of the transcarbamated material. Similarly, it is possible to perform further thermal steps, for example vacuum stripping, to remove organic volatiles from the carbamate-functional product, without unintended side reactions which are due to the presence of unreacted tin containing catalyst.

The transcarbamation reaction may provide a conversion of at least 75%, preferably 80% to 100%, very preferably 90 to 100% of theoretical total replacement of hydroxyl groups of the hydroxy-functional material with carbamate groups. In order to obtain a high degree of conversion, it is preferred if the formed by-product alcohol is removed during the transcarbamation reaction.

It is possible to react the hydroxyl groups of the hydroxy-functional material in the presence of the tin containing catalyst during preparation of a resin or during a polymerization reaction, in particular when the preparation step or polymerization does not depend on a reaction of the hydroxyl group. For example, in a last step of preparing the hydroxy-functional material, the alkyl carbamate of general formula (I) and tin containing catalyst could be charged to the reactor during such a final step to introduce the carbamate group upon formation of the hydroxyl group. Also in the case of polymerization of an addition copolymer, in which a monomer bearing hydroxyl groups, the alkyl carbamate of general formula (I) and tin containing catalyst can be introduced into the reactor before or with the hydroxyl monomer. This allows part or all of the transcarbamation to be completed by the time the initial monomer conversion is finished. The alkyl carbamate of general formula (I) and tin containing catalyst could also be introduced at a point during the time the monomer mixture is introduced into the reactor or after all of the monomers have been introduced into the reactor.

### Inventive Carbamate-Functional Material

The present invention is further directed to a carbamate-functional material prepared by the inventive method.

What has been said about the inventive method applies mutatis mutandis with respect to further preferred embodiments of the inventive carbamate-functional material.

### Inventive Coating Composition

The carbamate-functional material produced according to the inventive method can be used in coating compositions, preferably clear coating compositions. Said carbamate-functional material is preferably used as binder in said coating compositions. The term “binder” in the sense of the present invention and in agreement with DIN EN ISO 4618 (German version, date: March 2007), refers preferably to those curable nonvolatile fractions of the coating composition which are responsible for forming the film upon curing, with the exception of any pigments and fillers therein, and more particularly refers to the carbamate-functional resin and further resins optionally present which can be cured either by physical drying or by chemical crosslinking. Thus, the term “binder” in the sense of the present invention does not encompass curing agents or crosslinking agents used to crosslink the binders to effect film formation.

Such a coating composition may be cured by a reaction of the carbamate-functional material or materials with a curing agent that is a compound having a plurality of functional groups that are reactive with the carbamate groups of the material. Such reactive groups include active methylol, methylalkoxy or butylalkoxy groups on aminoplast crosslinking agents. An aminoplast is obtained by reaction of an activated nitrogen with a lower molecular weight aldehyde, optionally with further reaction with an alcohol (preferably a mono-alcohol with one to four carbon atoms such as methanol, isopropanol, n-butanol, isobutanol, etc.) to form an ether group. Preferred examples of activated nitrogens are activated amines such as melamine, benzoguanamine, cyclohexylcarboguanamine, and acetoguanamine; ureas, including urea itself, thiourea, ethyleneurea, dihydroxyethyleneurea, and guanylurea; glycoluril; amides, such as dicyandiamide; and carbamate functional compounds having at least one primary carbamate group or at least two secondary carbamate groups. The activated nitrogen is reacted with a lower molecular weight aldehyde. The aldehyde may be selected from formaldehyde, acetaldehyde, crotonaldehyde, benzaldehyde, or other aldehydes used in making aminoplast resins, although formaldehyde and acetaldehyde, especially formaldehyde, are preferred. The activated nitrogen groups are at least partially alkylolated with the aldehyde, and may be fully alkylolated; preferably the activated nitrogen groups are fully alkylolated. The reaction may be catalyzed by an acid.

The optional alkylol groups formed by the reaction of the activated nitrogen with aldehyde may be partially or fully etherified with one or more monofunctional alcohols. Suitable examples of the monofunctional alcohols include, without limitation, methanol, ethanol, n-propanol, isopropanol, n-butanol, isobutanol, tert-butyl alcohol, benzyl alcohol, and so on. Monofunctional alcohols having one to four carbon atoms and mixtures of these are preferred. The aminoplast may be at least partially etherified, and in various embodiments the aminoplast is fully etherified. For example, the aminoplast compounds may have a plurality of methylol and/or etherified methylol, butylol, or alkylol groups, which may be present in any combination and along with unsubstituted nitrogen hydrogens. Examples of suitable curing agent compounds include, without limitation, melamine formaldehyde resins, including monomeric or polymeric melamine resins and partially or fully alkylated melamine resins, and urea resins (e.g., methylol ureas such as urea formaldehyde resin, and alkoxy ureas such as butylated urea formaldehyde resin). One nonlimiting example of a fully etherified melamine-formaldehyde resin is hexamethoxymethyl melamine.

The alkylol groups are capable of self-reaction to form oligomeric and polymeric materials. Useful materials are characterized by a degree of polymerization. For melamine formaldehyde resins, it is preferred to use resins having a number average molecular weight less than about 2,000 g/mol, more preferably less than 1,500 g/mol, and even more preferably less than 1,000 g/mol.

Particularly preferred inventive coating compositions therefore comprise
a) at least one carbamate-functional material, very preferably at least one carbamate-functional polymer, said carbamate-functional material being prepared by reacting at least one alkyl carbamate compound of general formula (I)

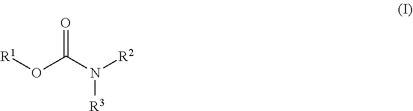

in which
   R¹ is an organic residue bearing at least one tertiary carbon atom and optionally at least one aryl group and/or at least one heteroatom;
   R², R³ are, independently from each other, selected from hydrogen or C₁ to C₁₀ alkyl groups,
   with at least one compound having at least one hydroxy group in the presence of at least one tin containing catalyst; and
b) optionally at least one curing agent.

The amount of the at least one carbamate-functional material (a) in the coating composition may be varied widely and is typically 5 to 45 wt. % solids, preferably 13 to 33 wt. % solids, based in each case on the total weight of the coating composition. Where preferred embodiments of the carbamate-functional material (a) are employed, the sum total of the weight-percentage fractions of all preferred embodiments of the carbamate-functional material (a) is preferably likewise 5 to 45 wt. % solids, very preferably 13 to 33 wt. % solids, based on the total weight of the coating composition.

A coating composition including the product carbamate-functional material(s) (a) and optionally at least one curing agent, preferably aminoplast curing agents, may further include a strong acid catalyst to enhance the crosslinking reaction. Such catalysts are well-known in the art and include, for example, para-toluenesulfonic acid, dinonylnaphthalene disulfonic acid, dodecylbenzenesulfonic acid, phenyl acid phosphate, monobutyl maleate, butyl phosphate, and hydroxy phosphate ester. Strong acid catalysts are often blocked, e.g. with an amine.

Apart from the at least one carbamate-functional material (a), the coating composition can comprise at least one further binder being different from carbamate-functional material (a). Suitable further binders include acrylics, vinyls, polyurethanes, polyurethane acrylate hybrid polymers, polycarbonates, polyesters, polyethers, alkyds, polysiloxanes, carbamate-functional acrylics or polyurethanes or aliphatic compounds and mixtures thereof.

A solvent may optionally be utilized in the coating compositions. Although the coating composition may be formulated, for example, in the form of a powder, it is often desirable that the composition be in a substantially liquid state, which can be accomplished with the use of a solvent to either dissolve or disperse the at least one carbamate-functional material and aminoplast crosslinker. In general, depending on the solubility characteristics of the components, the solvent can be any organic solvent and/or water, optionally with small amounts of organic water-soluble or -miscible co-solvents. The solvent in the coating composition is preferably present in an amount of from 0.01% to 99 wt. %, preferably 10 to 60 wt. %, and more preferably from 30 to 50 wt. %, based in each case on the total weight of the coating composition. Preferably, the solvent is an organic solvent. Suitable organic solvents are, for example, aliphatic hydrocarbons such as toluene, xylene, solvent naphtha, and mineral spirits, ketones such as acetone, cyclohexanone, methyl ethyl ketone or methyl amyl ketone, esters such as ethyl acetate, butyl acetate, pentyl acetate or ethyl ethoxy propionate, ethers such as glycol ethers like propylene glycol monomethyl ether, alcohols such as ethanol, propanol, isopropanol, n-butanol, isobutanol, and tert-butanol, nitrogen-containing compounds such as N-methyl pyrrolidone and N-ethyl pyrrolidone, or mixtures of the aforementioned solvents. Suitable organic solvent mixtures may be composed of aromatic hydrocarbons such as 1,2,4-trimethylbenzene, mesitylene, xylene, propylbenzene and isopropylbenzene or comprise aromatic hydrocarbons such as solvent naphtha.

When the coating compositions are formulated as basecoat topcoats, monocoat topcoats, tinted clearcoats or primers they contain pigments and optionally fillers, including special effect pigments. Nonlimiting examples of special effect pigments that may be utilized in basecoat and monocoat topcoat coating compositions include metallic, pearlescent, and color-variable effect flake pigments. Metallic (including pearlescent, and color-variable) topcoat colors are produced using one or more special flake pigments. Metallic colors are generally defined as colors having gonioapparent effects. For example, the American Society of Testing Methods (ASTM) document F284 defines metallic as “pertaining to the appearance of a gonioapparent material containing metal flake.” Metallic basecoat colors may be produced using metallic flake pigments like aluminum flake pigments, coated aluminum flake pigments, copper flake pigments, zinc flake pigments, stainless steel flake pigments, and bronze flake pigments and/or using pearlescent flake pigments including treated micas like titanium dioxide-coated mica pigments and iron oxide-coated mica pigments to give the coatings a different appearance (degree of reflectance or color) when viewed at different angles. Metal flakes may be cornflake type, lenticular, or circulation-resistant; micas may be natural, synthetic, or aluminum-oxide type. Flake pigments do not agglomerate and are not ground under high shear because high shear would break or bend the flakes or their crystalline morphology, diminishing or destroying the gonioapparent effects. The flake pigments are satisfactorily dispersed in a binder component by stirring under low shear. The flake pigment or pigments may be included in the coating composition in an amount of about 0.01 wt. % to about 0.3 wt. % or about 0.1 wt. % to about 0.2 wt. %, in each case based on total binder weight. Nonlimiting examples of commercial flake pigments include PALIOCROME® pigments, available from BASF Corporation.

Nonlimiting examples of other suitable pigments and fillers that may be utilized in basecoat and monocoat topcoat coating compositions include inorganic pigments such as titanium dioxide, barium sulfate, carbon black, ocher, sienna, umber, hematite, limonite, red iron oxide, transparent red iron oxide, black iron oxide, brown iron oxide, chromium oxide green, strontium chromate, zinc phosphate, silicas such as fumed silica, calcium carbonate, talc, barytes, ferric ammonium ferrocyanide (Prussian blue), and ultramarine, and organic pigments such as metallized and non-metallized azo reds, quinacridone reds and violets, perylene reds, copper phthalocyanine blues and greens, carbazole violet, monoarylide and diarylide yellows, benzimidazolone yellows, tolyl orange, naphthol orange, nanoparticles based on silicon dioxide, aluminum oxide or zirconium oxide, and so on. The pigment or pigments are preferably dispersed in a resin or polymer or with a pigment dispersant, such as binder resins of the kind already described, according to known methods. In general, the pigment and dispersing resin, polymer, or dispersant are brought into contact under a shear high enough to break the pigment agglomerates down to the primary pigment particles and to wet the surface of the pigment particles with the dispersing resin, polymer, or dispersant. The breaking of the agglomerates and wetting of the primary pigment particles are important for pigment stability and color development. Pigments and fillers may be utilized in amounts typically of up to about 60wt. %, based on total weight of the coating composition. The amount of pigment used depends on the nature of the pigment and on the depth of the color and/or the intensity of the effect it is intended to produce, and also by the dispersibility of the pigments in the pigmented coating composition. The pigment content, based in each case on the total weight of the pigmented coating composition, is preferably 0.5% to 50%, more preferably 1% to 30%, very preferably 2% to 20%, and more particularly 2.5% to 10wt. %.

Clearcoat coating compositions typically include no pigment but may include small amount of colorants or fillers that do not unduly affect the transparency or desired clarity of the clearcoat coating layer produced from the composition.

Additional desired, customary coating additives agents may be included, for example, surfactants, stabilizers, wetting agents, dispersing agents, adhesion promoters, UV absorbers, hindered amine light stabilizers such as HALS compounds, benzotriazoles or oxalanilides; free-radical scavengers; slip additives; defoamers; reactive diluents, of the kind which are common knowledge from the prior art; wetting agents such as siloxanes, fluorine compounds, carboxylic monoesters, phosphoric esters, polyacrylic acids and their copolymers, for example polybutyl acrylate, or polyurethanes; adhesion promoters such as tricyclodecanedimethanol; flow control agents; film-forming assistants such as cellulose derivatives; rheology control additives; crosslinked polymeric microparticles; inorganic phyllosilicates such as aluminum-magnesium silicates, sodium-magnesium and sodium-magnesium-fluorine-lithium phyllosilicates of the montmorillonite type; silicas such as Aerosils®; or synthetic polymers containing ionic and/or associative groups such as polyvinyl alcohol, poly(meth)acrylamide, poly(meth)acrylic acid, polyvinylpyrrolidone, styrene-maleic anhydride copolymers or ethylene-maleic anhydride copolymers and their derivatives, or hydrophobically modified ethoxylated urethanes or polyacrylates; flame retardant; and so on. Typical coating composition include one or a combination of such additives in customary amounts, for example from 0.1 to 20 wt. %, based in each case on the total weight of the coating composition.

What has been said about the inventive method and the inventive carbamate-functional material applies mutatis mutandis with respect to further preferred embodiments of the inventive coating composition.

### Inventive Method to at Least Partly Coat a Substrate

In the inventive method, an inventive coating composition is at least partially applied on a substrate and cured.

Application to the substrate can be performed by any of a number of techniques well-known in the art. These include, for example, spray coating, dip coating, roll coating, curtain coating, and the like. For automotive body panels, spray coating is preferred. The coating compositions of the invention can be applied by any of the typical application methods, such as spraying, knife coating, spreading, pouring, dipping, impregnating, trickling or rolling, for example. In the course of such application, the substrate to be coated may itself be at rest, with the application equipment or unit being moved. Alternatively, the substrate to be coated, in particular a coil, may be moved, with the application unit at rest relative to the substrate or being moved appropriately. Preference is given to employing spray application methods, such as compressed-air spraying, airless spraying, high-speed rotation, electrostatic spray application, alone or in conjunction with hot spray application such as hot-air spraying, for example.

The coating compositions and coating systems of the invention, especially the clearcoat systems, are employed in particular in the technologically and esthetically particularly demanding field of automotive OEM finishing and also of automotive refinish. With particular preference the coating compositions of the invention are used in multistage coating methods, particularly in methods where a pigmented basecoat film is first applied to an uncoated or precoated substrate and thereafter a film with the coating compositions of the invention is applied. The invention, accordingly, also provides multicoat effect and/or color coating systems comprising at least one pigmented basecoat and at least one clearcoat disposed thereon, wherein the clearcoat has been produced from the inventive coating composition containing the carbamate-functional material(s) as disclosed herein.

When the coating composition is used as the clearcoat of a composite color-plus-clear coating, the pigmented basecoat composition may be a coating composition containing the carbamate-functional material(s) produced according to the inventive method or may be any of a number of types well-known in the art, and does not require explanation in detail herein. Polymers known in the art to be useful in basecoat compositions include acrylics, vinyls, polyurethanes, polyurethane acrylate hybrid polymers, polycarbonates, polyesters, polyethers, alkyds, polysiloxanes and mixtures thereof. Preferred polymers include acrylics, polyurethanes, polyurethane acrylate hybrid polymers, polyesters and mixtures thereof. In one preferred embodiment of the invention, the basecoat composition also utilizes a carbamate-functional acrylic polymer. Basecoat polymers may be thermoplastic, but are preferably crosslinkable and comprise one or more type of crosslinkable functional groups. Such groups include, for example, hydroxy, isocyanate, amine, epoxy, acrylate, vinyl, silane, and acetoacetate groups. These groups may be masked or blocked in such a way so that they are unblocked and available for the crosslinking reaction under the desired curing conditions, generally elevated temperatures. Basecoat polymers may be self-crosslinkable or may require a separate crosslinking agent that is reactive with the functional groups of the polymer. When the polymer comprises hydroxy functional groups, for example, the crosslinking agent may be an aminoplast resin, isocyanate and blocked isocyanates (including isocyanurates), and acid or anhydride functional crosslinking agents.

Not only water-thinnable basecoat materials but also basecoat materials based on organic solvents can be used. The coating composition of the invention is applied after a film of the basecoat material has been formed on the substrate. After application, a certain rest time or “flash” period can be performed. The rest time serves, for example, for the leveling and devolatilization of the coating films or for the evaporation of volatile constituents such as solvents. The rest time may be assisted or shortened by the application of elevated temperatures or by a reduced humidity, provided this does not entail any damage or alteration to the coating films, such as premature complete crosslinking, for instance. Curing is preferably performed under conditions employed for automotive OEM finishing, at temperatures from 30 to 200° C., more preferably 40 to 190° C., and in particular 50 to 180° C., for a time of 1 min up to 10 h, more preferably 2 min up to 5 h, and in particular 3 min to 3 h, although longer curing times may also be employed at the temperatures used for automotive refinish, which are preferably between 30 and 90° C. Curing can be effected by thermal curing in accordance with the typical, known methods such as heating in a forced-air oven or irradiation with IR lamps. The thermal cure may also take place in stages. Another preferred curing method is that of curing with near infrared (NIR) radiation. Although various methods of curing may be used, heat-curing is preferred. Generally, heat curing is effected by exposing the coated article to elevated temperatures provided primarily by radiative heat sources

The cured basecoat layers formed may have a thickness of from 5 to 75 μm, depending mainly upon the color desired and the thickness needed to form a continuous layer that will provide the color. The cured clearcoat layers formed typically have thicknesses of from 30 μm to 65 μm.

The coating composition can be applied onto many different types of substrates, including metal substrates such as bare steel, phosphated steel, galvanized steel, or aluminum; and non-metallic substrates, such as plastics and composites. The substrate may also be any of these materials having upon it already a layer of another coating, such as a layer of an electrodeposited primer, primer surfacer, and/or basecoat, cured or uncured.

The substrate may be first primed with an electrodeposition (electrocoat) primer. The electrodeposition composition can be any electrodeposition composition used in automotive vehicle coating operations. Non-limiting examples of electrocoat compositions include the CATHOGUARD® electrocoating compositions sold by BASF Corporation. Electrodeposition coating baths usually comprise an aqueous dispersion or emulsion including a principal film-forming epoxy resin having ionic stabilization (e.g., salted amine groups) in water or a mixture of water and organic cosolvent. Emulsified with the principal film-forming resin is a crosslinking agent that can react with functional groups on the principal resin under appropriate conditions, such as with the application of heat, and so cure the coating. Suitable examples of crosslinking agents include, without limitation, blocked polyisocyanates. The electrodeposition coating compositions usually include one or more pigments, catalysts, plasticizers, coalescing aids, antifoaming aids, flow control agents, wetting agents, surfactants, UV absorbers, HALS compounds, antioxidants, and other additives.

The electrodeposition coating composition is preferably applied to a dry film thickness of 10 to 35 μm. After application, the coated vehicle body is removed from the bath and rinsed with deionized water. The coating may be cured under appropriate conditions, for example by baking at 135° C. to 190° C. for 15 to 60 minutes.

Because the coatings of the invention produced from the coating compositions of the invention adhere excellently even to electrocoats, surfacer coats, basecoat systems or typical, known clearcoat systems that have already been cured, they are outstandingly suitable not only for use in automotive OEM finishing but also for automotive refinish or for the modular scratch proofing of automobile bodies that have already been painted.

What has been said about the inventive method, the inventive carbamate-functional material and the inventive coating composition applies mutatis mutandis with respect to further preferred embodiments of the inventive coating method.

The invention is described in particular by the following embodiments:

Embodiment 1: method for preparing a carbamate-functional material, wherein at least one alkyl carbamate compound of general formula (I)

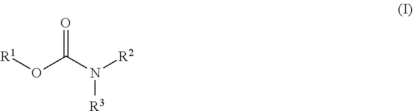

in which
R¹ is an organic residue bearing at least one tertiary carbon atom and optionally at least one aryl group and/or at least one heteroatom;
R², R³ are, independently from each other, selected from hydrogen or C₁ to C₁₀ alkyl groups,
is reacted with at least one compound having at least one hydroxy group in the presence of at least one tin containing catalyst.

Embodiment 2: method according to embodiment 1, wherein R¹ in general formula (I) is selected from aliphatic tertiary C₄ to C₁₂ alkyl residues, preferably aliphatic tertiary C₄ to C₁₀ alkyl residues, more preferably aliphatic tertiary C₄ to C₆ alkyl residues, very preferably aliphatic tertiary C₄ residues.

Embodiment 3: method according to embodiment 1 or 2, wherein R² and R³ in general formula (I), are selected from hydrogen.

Embodiment 4: method according to any of the preceding embodiments, wherein the at least one alkyl carbamate compound of general formula (I) is tert-butyl carbamate.

Embodiment 5: method according to any of the preceding embodiments, wherein the at least one compound having at least one hydroxy group has an average OH-functionality of 1.5 to 10, preferably 1.8 to 8, more preferably 1.8 to 6, very preferably 2 to 4.

Embodiment 6: method according to any of the preceding embodiments, wherein the at least one compound having at least one hydroxy group is selected from polyols having 2 to 160 carbon atoms, polyester polyols, polyhydroxy polycarbonates, polyether polyols, polyurethane polyols, polyvinyl polymer polyols, polyhydroxy polyesteramides, polysiloxane polyols, polyhydroxy polythioethers, hyperbranched polyols and mixtures thereof, preferably polyester polyols and/or polyhydroxy polycarbonates.

Embodiment 7: method according to embodiment 6, wherein the polyester polyol is a reaction product of epsilon caprolactone and pentaerythritol.

Embodiment 8: method according to embodiment 6 or 7, wherein the polyester polyol has a weight average molecular weight M_{w} of 500 to 2,000 g/mol, preferably 600 to 1,500 g/mol, very preferably 900 to 1,200 g/mol, as determined according to gel permeation chromatography using polystyrene as internal standard.

Embodiment 9: method according to any of embodiments 6 to 8, wherein the polyester polyol has a hydroxyl number of 150 to 400 mg KOH/g solids, preferably 180 to 350 mg KOH/g solids, more preferably 200 to 300 mg KOH/g solids, very preferably 215 to 235 mg KOH/g solids, as determined according to R.-P. Krüger, R. Gnauck and R. Algeier, Plaste und Kautschuk, 20, 274 (1982).

Embodiment 10: method according to any of embodiments 6 to 9, wherein the polyhydroxy polycarbonate is prepared by reacting carbon acid compounds, preferably diphenyl carbonate and/or dimethyl carbonate and/or phosgene, with diols, preferably 1,5-pentane diol and/or 1,6-hexane diol.

Embodiment 11: method according to any of embodiments 6 to 10, wherein the polyhydroxy polycarbonate has a weight average molecular weight M_{w} of 500 to 3,000 g/mol, preferably 700 to 2,500 g/mol, very preferably 950 to 2,000 g/mol, as determined according to gel permeation chromatography using polystyrene as internal standard.

Embodiment 12: method according to any of embodiments 6 to 11, wherein the polyhydroxy polycarbonate has a hydroxyl number of 30 to 300 mg KOH/g solids, preferably 35 to 200 mg KOH/g solids, more preferably 40 to 150 mg KOH/g solids, very preferably 50 to 65 mg KOH/g solids or 100 to 120 mg KOH/g solids, as determined according to R.-P. Krüger, R. Gnauck and R. Algeier, Plaste und Kautschuk, 20, 274 (1982).

Embodiment 13: method according to any of the preceding embodiments, wherein a molar ratio of the at least one alkyl carbamate compound of general formula (I), preferably tert-butyl carbamate, to the at least one compound having at least one hydroxy group, preferably polyester polyol and/or polyhydroxy polycarbonate, of 1.5 to 5, preferably 1.5 to 3, very preferably 1.5 to 2, is used.

Embodiment 14: method according to any of the preceding embodiments, wherein the at least one tin containing catalyst is selected from dialkyltin alkanoates, dialkyl tin oxides and mixtures thereof, preferably dibutyltin oxide.

Embodiment 15: method according to any of the preceding embodiments, wherein the at least one tin containing catalyst, preferably dibutyltin oxide, is used in a total amount of 0.01 to 1 wt. %, preferably 0.02 to 0.5 wt. %, more preferably 0.03 to 0.3 wt. %, very preferably 0.05 to 0.2 wt. %, based in each case on the total amount of the at least one alkyl carbamate compound of general formula (I) and the at least one compound having at least one hydroxy group.

Embodiment 16: method according to any of the preceding embodiments, wherein the reaction is carried out at a temperature in the range of from 110° C. to 140° C.

Embodiment 17: method according to any of the preceding embodiments, wherein the reaction is carried out in an organic solvent, said solvent being preferably selected from aromatic hydrocarbons, preferably toluene, xylene, mesitylene, 2-, 3-, or 4-ethyltoluene; naphthas; aliphatic and cycloaliphatic hydrocarbons, preferably white spirits, cyclohexane, mineral turpentine, tetralin and decalin; ketones, and mixtures thereof, preferably toluene.

Embodiment 18: method according to any of the preceding embodiments, wherein at least 75%, preferably 80 to 100%, very preferably 90 to 100%, of the hydroxy groups present in the at least one compound comprising at least one hydroxy group are replaced with carbamate groups.

Embodiment 19: carbamate-functional material prepared by a method acceding to any of embodiments 1 to 18.

Embodiment 20: coating composition comprising at least one carbamate-functional material according to embodiment 19.

Embodiment 21: coating composition according to embodiment 20, wherein the coating composition is a clearcoat composition or a tinted clearcoat composition, preferably a clearcoat composition.

Embodiment 22: method of coating a substrate, comprising applying a coating composition according to embodiment 20 or 21 at least partly to the substrate and curing said applied coating composition.

### EXAMPLES

The present invention will now be explained in greater detail through the use of working examples, but the present invention is in no way limited to these working examples. Moreover, the terms “parts”, “%” and “ratio” in the examples denote “parts by mass”, “mass %” and “mass ratio” respectively unless otherwise indicated.

### 1. Methods of Determination

### 1.1 Number-Average Molecular Weight (Mₙ), Weight-Average Molecular Weight (M_{w})

The number-average molecular weight distribution (Mₙ) and the weight-average molecular weight distribution (M_{w}) were, unless otherwise indicated, determined by GPC-analysis (gel permeation chromatography analysis) according to DIN 55672-1:2007-08 at 40° C. using a high-pressure liquid chromatography pump. The eluent used for the copolymers was tetrahydrofuran with an elution rate of 1 ml/min. The calibration was done with very narrow distributed polystyrene standards from the Polymer Laboratories with a molecular weight M=from 580 until 6.870.000 g/mol.

### 1.2 Determination of OH-Number

The hydroxyl number was determined on the basis of R.-P. Krüger, R. Gnauck and R. Algeier, Plaste and Kautschuk, 20, 274 (1982), by means of acetic anhydride in the presence of 4-dimethylaminopyridine as a catalyst in a tetrahydrofuran (THF)/dimethylformamide (DMF) solution at room temperature, by fully hydrolyzing the excess of acetic anhydride remaining after acetylation and conducting a potentiometric back-titration of the acetic acid with alcoholic potassium hydroxide solution. Acetylation times of 60 minutes were sufficient in all cases to guarantee complete conversion.

### 1.3 ¹H-NMR

¹H-NMR measurements were conducted at 300 MHz using D⁶-DMSO as solvent.

### 2. Transcarbamation of Different Hydroxy-Functional Compounds

### 2.1 Example 1 of the Invention: Transcarbamation of a Polyhydroxy Polycarbonate using a Tertiary Alkyl Carbamate of General Formula (I)

A dicarbamate was prepared from a polyhydroxy polycarbonate using tert-butyl carbamate and dibutyltin oxide as follows:

A mixture of 93.23 parts of a polyhydroxy polycarbonate A (commercial product Ravecarb 103 (copolymer from hexanediol and pentanediol) having a M_{w} of 3,052 g/mol, a Mₙ of 1,667 g/mol (determined according to point 1.1) and an OH-number of 111 mg KOH/g solids), 23.77 parts of tert-butyl carbamate (alkyl carbamate of general formula (I) with R¹=tert-butyl group, R²=R³=hydrogen), 0.11 parts dibutyltin oxide and 208.55 parts of toluene was heated under an inert atmosphere to reflux in a reactor equipped with an extractor that can remove the azeotrope of formed tert-butanol and toluene. Once at reflux, the inert atmosphere was turned off. The reaction was ended when approximately 95% of the hydroxyl groups present in the polycarbonate were converted to carbamate groups (around 7 to 8 hours from beginning of reflux). Unreacted tert-butyl carbamate and toluene were removed by distillation to obtain a viscous liquid. The percent of conversion was calculated to be 95.3%, based on the final OH-number of the carbamate-functional material of 5.18 mg KOH/g solids. The theoretical carbamate equivalent weight of this product is 530.4. The product was used as is.

Molecular weight of obtained carbamate-functional material determined according to point 1.1: Mₙ=1,506 g/mol, M_{w}=2,950 g/mol. ¹H-NMR values (proton account is normalized per chain): δ1.29-1.30 (m), 1.49-1.65 (m), 3.85-3.90 (m, 4H), 4.03-4.08 (m), 6.41 (br, 4H).

### 2.2 Example 2 of the Invention: Transcarbamation of a further Polyhydroxy Polycarbonate using a Tertiary Alkyl Carbamate of General Formula (I)

Transcarbamation was performed as described in point 2.1 using 507.53 parts of a polyhydroxy polycarbonate B (commercial product Ravecarb 107 (copolymer from hexanediol and pentanediol) having a M_{w} of 6,489 g/mol, a Mn of 3,202 g/mol (determined according to point 1.1.) and an OH-number of 56 mg KOH/g solids), 65.30 parts of tert-butyl carbamate, 0.30 parts of dibutyltin oxide and 365.21 parts of toluene. A colorless viscous liquid was obtained. The percent of conversion was calculated to be 95.0%, based on the final OH-number of the carbamate-functional material of 2.80 mg KOH/g solids. The theoretical carbamate equivalent weight of this product is 1054.7. The product was then diluted with cyclohexanone to obtain 82 wt. % resin.

Molecular weight of obtained carbamate-functional material determined according to point 1.1: Mₙ=2,847 g/mol, M_{w}=6,581 g/mol.

¹H-NMR values (proton account is normalized per chain): δ1.26-1.39 (m), 1.50-1.70 (m), 3.85-3.90 (m, 4H), 4.02-4.07 (m), 6.41 (br, 4H).

### 2.3 Example 3 of the Invention: Transcarbamation of a Hydroxy-Functional Polyester using a Tertiary Alkyl Carbamate of General Formula (I)

Transcarbamation was performed as described in point 2.1 using 262.34 parts of a hydroxyfunctional polyester (commercial product Capa® 4101 (reaction product of epsilon-caprolactone and pentaerythritol) having a M_{w} of 1,804 g/mol, a Mₙ of 1,471 g/mol (determined according to point 1.1) and an OH-number of 226 mg KOH/g solids), 133.75 parts of tert-butyl carbamate, 0.69 parts of dibutyltin oxide and 250.00 parts of toluene. A colorless viscous liquid was obtained. The percent of conversion was calculated to be 91.4%, based on the final OH-number of the carbamate-functional material of 19.51 mg KOH/g solids. The theoretical carbamate equivalent weight of this product is 314.7. The product was then diluted with cyclohexanone to obtain 80 wt. % resin.

Molecular weight of obtained carbamate-functional material determined according to point 1.1: Mₙ=1,800 g/mol, M_{w}=2,513 g/mol.

¹H-NMR values (proton account is normalized per chain): δ1.22-1.38 (m, 4H), 1.44-1.64 (m, 8H), 2.28 (t, 4H), 3.87 (t, 2H), 3.99 (t, 2H), 4.05 (t, 2H), 6.25-6.71 (br, 2H)

### 2.4 Comparative Example 1: Transcarbamation of a Polyhydroxy Polycarbonates using an Alkyl Carbamate

The transcarbamation described in point 2.1 was repeated. Instead of tert-butyl carbamate, the corresponding amount of methyl carbamate was used. The degree of conversion was 96.7%.

Molecular weight of the obtained carbamate-functional material determined according to point 1.1: Mₙ=1,370 g/mol, M_{w}=2,539 g/mol.

### 2.5 Comparative Example 2: Transcarbamation of a Polyhydroxy Polycarbonates using an Alkyl Carbamate

The transcarbamation described in point 2.2 was repeated. Instead of tert-butyl carbamate, the corresponding amount of methyl carbamate was used.

### 2.6 Comparative Example 3: Transcarbamation of a Hydroxy-Functional Polyester using an Alkyl Carbamate

The transcarbamation described in point 2.3 was repeated. Instead of tert-butyl carbamate, the corresponding amount of methyl carbamate was used. The degree of conversion was 97.7%.

Molecular weight of the obtained carbamate-functional material as determined according to point 1.1: Mₙ=1,433 g/mol, M_{w}=2,087 g/mol.

### 3. Results Obtained from Inventive and Comparative Transcarbamation Reactions

Table 1 lists the results with respect to molecular weight of the carbamate-functional materials obtained from the inventive and comparative transcarbamation reactions previously described.

TABLE 1
Molecular weight M_{w} of prepared carbamate-functional materials

| | Carbamate | Hydroxy-functional | Molecular weight |
|---|---|---|---|
| Example | compound | material | M_{w} [g/mol] |
| Inventive | tert-butyl | Polyhydroxy | 2,950 |
| example 1 | carbamate | polycarbonate A | |
| Inventive | tert-butyl | Polyhydroxy | 6,581 |
| example 2 | carbamate | polycarbonate B | |
| Inventive | tert-butyl | Hydroxy-functional | 2,513 |
| example 3 | carbamate | polyester | |
| Comparative | Methyl | Polyhydroxy | 2,539 |
| example 1 | carbamate | polycarbonate A | |
| Comparative | Methyl | Polyhydroxy | n.d. ¹⁾ |
| example 2 | carbamate | polycarbonate B | |
| Comparative | Methyl | Hydroxy-functional | 2,087 |
| example 3 | carbamate | polyester | |

¹⁾ not determined

As is apparent from Table 1, the use of methyl carbamate results in a 15 to 17wt. % loss of the produced carbamate-functional material (see comparative examples 1 to 3) compared to the use of tert-butyl carbamate (see inventive examples 1 to 3). Without wishing to be bound to this theory, it is believed that this undesired weight loss is due to the reaction of the by-product methanol formed when using methyl carbamate with the carbonate or ester functions present in the hydroxy-functional material. Said undesired side reaction is leading to a cleavage of the backbone of the hydroxy-functional material and is not observed when using tert-butyl carbamate (see inventive examples 1 to 3). The inventive method therefore provides carbamate-functional compounds in high yields and purities without undesired side reactions and can be used on a large variety of hydroxy-functional compounds comprising further functional groups, like carbonate or ester groups.

### 4. Coating Compositions Comprising Carbamate-Functional Materials

The clearcoat compositions listed in Table 2 were prepared by mixing and stirring the ingredients until a homogenous clearcoat composition is obtained.

### 4.1 Synthesis Procedure for Carbamate-Functional Acrylic Resin

A reactor flushed with nitrogen and fitted with a condenser was charged with 808.8 parts of glycidyl neodecanoate, 381.5 parts of Solvesso® 100 and 429.8 parts of methylcarbamate, and this initial charge was heated to 142 ° C. with stirring. To this reaction mixture, a mixture of 280.2 parts of methacrylic acid, 203.8 parts of cyclohexyl methacrylate, 744.6 parts of 2-hydroxyethyl methacrylate, 182.3 parts of 2,2′-azobis(2-methylbutyronitrile) (VAZO 67), 312.8 parts of toluene, and 82.1 parts of Solvesso® 100 were metered in at a uniform rate over a time of 240 minutes. After the addition was completed, the reaction mixture was held at 142° C. for 45 minutes. 14.8 parts of 2,2′-azobis(2-methylbutyronitrile) (VAZO 67) and 25.4 parts of toluene were metered in at a uniform rate over a time of 60 minutes, and the reaction mixture was held at 142° C. for 60 minutes after the addition was completed. To this reaction mixture, 7.17 parts of FASTCAT® 4100 was added, and the reaction mixture was heated under an inert atmosphere to reflux allowing removal of the azeotrope of methanol and toluene. Once at reflux, the inert atmosphere was turned off, and 282.5 parts of toluene was slowly added to the reactor over the course of the transcarbamation to offset the loss of toluene and to maintain the reflux temperature to be <142° C. The reaction was stopped when more than 90% hydroxyl group was converted to carbamate group. Free methylcarbamate and the solvent were removed by vacuum distillation, and a mixture of Solvesso® 100 and methoxy propanol was added to adjust the solid content (% NV) of the resin to be 65.0 wt. %

### 4.2 Coating Compositions

TABLE 2
Ingredients of comparative clearcoat composition CC-C1 and inventive
clearcoat compositions CC-I1 to CC-I3 (amounts in wt. %, based
on the total weight of the respective clearcoat composition)

| Ingredient | CC-C1 | CC-I1* | CC-I2* | CC-I3* |
|---|---|---|---|---|
| Carbamate functional acrylic | 21.2 | 7.65 | 6.06 | 8.28 |
| resin ¹⁾ | | | | |
| Carbamate-functional material of | — | 23.2 | — | — |
| inventive example 1 | | | | |
| Carbamate-functional material of | — | — | 39.2 | — |
| inventive example 2 | | | | |
| Carbamate-functional material of | — | — | — | 16.9 |
| inventive example 3 | | | | |
| Carbamate functional oligomer ²⁾ | 16.4 | 14.4 | 11.4 | 15.5 |
| Epoxy acrylic resin ³⁾ | 1.76 | 1.54 | 1.22 | 1.66 |
| Melamine resin ⁴⁾ | 10.1 | 9.03 | 7.14 | 9.76 |
| Isocyanate crosslinker ⁵⁾ | 2.82 | 2.47 | 1.95 | 2.67 |
| UV absorber ⁶⁾ | 4.26 | 3.73 | 2.95 | 4.04 |
| HALS ⁷⁾ | 0.533 | 0.467 | 0.370 | 0.505 |
| Rheology Agent 1 ⁸⁾ | 15.2 | 13.3 | 10.5 | 14.4 |
| Rheology Agent 2 ⁹⁾ | 15.1 | 13.3 | 10.5 | 14.3 |
| Rheology Agent 3 ¹⁰⁾ | 0.212 | 0.186 | 0.147 | 0.201 |
| anti-popping additive ¹¹⁾ | 0.024 | 0.021 | 0.017 | 0.023 |
| flow additive ¹²⁾ | 0.150 | 0.132 | 0.104 | 0.143 |
| Tin Catalyst ¹³⁾ | 0.195 | 0.171 | 0.135 | 0.184 |
| Acid Catalyst ¹⁴⁾ | 2.95 | 2.58 | 2.04 | 2.79 |
| Solvent ¹⁵⁾ | 9.10 | 7.98 | 6.31 | 8.63 |
| Total non-volatile content [%] | 53.5 | 62.6 | 63.3 | 56.1 |
| Total carbamate equivalent | 1,262 | 1,051 | 1,406 | 1,017 |

*inventive
¹⁾ see point 4.1
²⁾ Blend of 50% of solid weight of the reactive component (a) described in U.S. Pat. No. 6,962,730 B2 and 50% of solid weight of the resin described in example 1 of U.S. Pat. No. 5,719,237 A
³⁾ weight per epoxy = 381 g/mol, solids = 59.4%, T_{g} = −31° C., monomer composition (wt. %, based on total weight of epoxy acrylic resin): sty/MMA/HPMA/HPA/GMA/EHA/acetic anhydride = 0.971/0.971/1.85/1.85/31.8/56.2/2.13
⁴⁾ Resimene 747 (supplied by Ineos)
⁵⁾ DESMODUR PL 350 MPA/SN (supplied by Covestro)
⁶⁾ Tinuvin ® 928 (supplied by BASF SE)
⁷⁾ Tinuvin ® 123 (supplied by BASF SE)
⁸⁾ 28.9% silica in acrylic resin (M_{w} = 4,600 g/mol, hydroxyl number = 182 mg KOH/g solids, solids = 67.5%, T_{g} = 34° C., monomer composition (wt. %, based on the total weight of the acrylic resin): MAA/HPMA/EHMA/EHA/CHMA = 0.419/46.2/23.3/11.1/19.0))
⁹⁾ 2.72% Diurea crystals in carbamate functional acrylic resin (see point 4.1)
¹⁰⁾ BYK-LP R 23429 (solution of polyhydroxycarboxylic acid amides)
¹¹⁾ Flowlen AC-300 (supplied by Kyoeisha Chemical)
¹²⁾ Lindron 22 PolyButyl Acrylate (commercially available from Lindau Chemicals)
¹³⁾ Fascat ® 4200 (supplied by PMC Organometallix)
¹⁴⁾ mixture of carboxylic acid, sulfonic acid and blocked sulfonic acid
¹⁵⁾ mixture of EXXAL 13, ethyl 3-ethoxyproprionate, n-butanol, Dowanol PM, amyl acetate

### 5. Preparation of Coated Substrates and Evaluation of Clearcoat Layer

### 5.1 Preparation of Coated Substrates

Zinc-plated steel plates (0.8 mm thick, 304.8 mm long and 101.6 mm wide) were chemically treated with zinc phosphate, electrocoated using the cationic electrocoat paint Cathoguard® 800 (BASF Japan (Co.)) to give a dry film thickness of 25 μm, and baked for 30 minutes at 170° C. A primer coat [solvent borne light gray primer (U28AU227F) commercially available from BASF Japan (Co.)] was spray applied to give a dry film thickness of 23 to 27 μm, flashed for 10 minutes at 23° C. and baked for 15 minutes at 155° C. Next, a basecoat paint (solvent-borne high solids basecoat Shadow Black (E387KU343C) commercially available from BASF Japan (Co.)) was spray-coated to give a dry film thickness of 17 to 23 μm, and was dried for 10 minutes at 23° C. Afterwards, the clear coating compositions CC-I1 to CC-I3 according Table 2 were each applied wet-in-wet by spray-coating to the basecoat film to give a dry film thickness of 45 to 51 μm. After maintaining the panels for 10 minutes at 23° C., the samples were baked for 25 minutes at 140° C. to obtain the substrates coated with a multilayer coating. Afterwards, the properties of each clearcoat layer were determined with the methods described hereinafter.

### 5.2 Evaluation of Clearcoat Layer—Dry Scratch Resistance

The clearcoat layer of the formed multilayer coating was evaluated for dry scratch resistance as follows:

Dry scratch resistance was carried out with a crock meter (M238BB Electronic Crockmeter, SDL Atlas) equipped with a micro-scratch head having a width of 25 mm (±0.5 mm) and a curvature with a radius of 19 mm (±0.5 mm). The micro-scratch head is first covered with black EPDM open cell foam with a Shore 00 hardness of 60±5, and is then covered with an 5 μm Aluminum Oxide Lapping Polyester film (261X, lot#2533-5, from 3M). The applied force to the panel is around 9 N, the length of the scratch line is 11 cm and the speed of the crockmeter is 1 Hz. The movement of the micro scratch head relative to the panel is perpendicular to the axis of the surface curvature.

The initial gloss of the samples (20°) was measured perpendicularly to the length of the panels (which will also be the scratching direction) on 3 panels at 4 locations evenly distributed on each panel, the average value for each panel being reported as initial gloss value. Gloss was measured with a gloss-meter (micro-tri-gloss, BYK-10 GARDENER). Five back and forth (double stroke) movement were performed with the crock meter, while correctly maintaining the panel on the device. Two new scratches were made on each of the 3 panels. Gloss after scratching (20°) is measured immediately, 24 h±30 min, and 168 h±1 h, after each individual scratch line is made. Samples are stored at ambient temperature (23° C.). The average value of all post scratch measurements for each panel and each scratch line being reported as post-scratch value. The average gloss retention for each scratch line of each panel is calculated by dividing the post-scratch value by the initial gloss value of the respective panel. The average gloss retention is afterwards obtained by dividing the average gloss retention of all scratch lines by the average initial gloss value obtained from the initial gloss measurements on each panel.

Gloss Retention in %=(average gloss retention of all scratch lines/average initial gloss)×100%

### 6. Results

The obtained results for the clearcoat layers produced according to point 5.1 are shown in Table 3.

TABLE 3
Dry scratch resistance of clearcoat layers obtained
from inventive clearcoat compositions CC-I1 to CC-I3
and comparative clearcoat composition CC-C1 (see
Table 2 for description of footnotes 1) to 15))

| | Multilayer coating | | | |
|---|---|---|---|---|
| | MC-1 | MC-2* | MC-3* | MC-4* |
| Clearcoat composition | CC-C1 | CC-I1 | CC-I2 | CC-I3 |
| Carbamate functional acrylic | 21.2 | 7.65 | 6.06 | 8.28 |
| resin ¹⁾ | | | | |
| Carbamate-functional material of | − | 23.2 | − | − |
| inventive example 1 | | | | |
| Carbamate-functional material of | − | − | 39.2 | − |
| inventive example 2 | | | | |
| Carbamate-functional material of | − | − | − | 16.9 |
| inventive example 3 | | | | |
| Carbamate functional oligomer ²⁾ | 16.4 | 14.4 | 11.4 | 15.5 |
| Epoxy acrylic resin ³⁾ | 1.76 | 1.54 | 1.22 | 1.66 |
| Melamine resin ⁴⁾ | 10.1 | 9.03 | 7.14 | 9.76 |
| Isocyanate crosslinker ⁵⁾ | 2.82 | 2.47 | 1.95 | 2.67 |
| UV absorber ⁶⁾ | 4.26 | 3.73 | 2.95 | 4.04 |
| HALS ⁷⁾ | 0.533 | 0.467 | 0.370 | 0.505 |
| Rheology Agent 1 ⁸⁾ | 15.2 | 13.3 | 10.5 | 14.4 |
| Rheology Agent 2 ⁹⁾ | 15.1 | 13.3 | 10.5 | 14.3 |
| Rheology Agent 3 ¹⁰⁾ | 0.212 | 0.186 | 0.147 | 0.201 |
| anti-popping additive ¹¹⁾ | 0.024 | 0.021 | 0.017 | 0.023 |
| flow additive ¹²⁾ | 0.150 | 0.132 | 0.104 | 0.143 |
| Tin Catalyst ¹³⁾ | 0.195 | 0.171 | 0.135 | 0.184 |
| Acid Catalyst ¹⁴⁾ | 2.95 | 2.58 | 2.04 | 2.79 |
| Solvent ¹⁵⁾ | 9.10 | 7.98 | 6.31 | 8.63 |
| Gloss retention [%] | 78.6 | 85.2 | 83.3 | 82.3 |

*inventive

### 7. Discussion of Results

The carbamate-functional materials prepared according to the invention can be used as binders in clearcoat compositions and provide multilayer coatings having an improved gloss retention (see MC-2 to MC-4) compared to multilayer coating MC-1, being prepared from a clearcoat comprising different carbamate-functional materials.

1. A method for preparing a carbamate-functional material, wherein at least one alkyl carbamate compound of general formula (I) 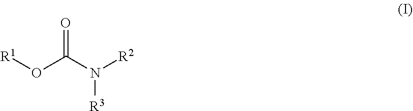
in which
R¹ is an organic residue bearing at least one tertiary carbon atom and optionally at least one aryl group and/or at least one heteroatom;
R², R³ are, independently from each other, selected from the group consisting of hydrogen and C₁ to C₁₀ alkyl groups, and
is reacted with at least one compound having at least one hydroxy group in the presence of at least one tin containing catalyst.

2. The method according to claim 1, wherein R¹ in general formula (I) is selected from the group consisting of aliphatic tertiary C₄ to C₁₂ alkyl residues.

3. The method according to claim 1, wherein R² and R³ in general formula (I) are selected from hydrogen.

4. The method according to claim 1, wherein the at least one alkyl carbamate compound of general formula (I) is tert-butyl carbamate.

5. The method according to claim 1, wherein the at least one compound having at least one hydroxy group has an average OH-functionality of 1.5 to 10.

6. The method according to claim 1, wherein the at least one compound having at least one hydroxy group is selected from the group consisting of polyols having 2 to 160 carbon atoms, polyester polyols, polyhydroxy polycarbonates, polyether polyols, polyurethane polyols, polyvinyl polymer polyols, polyhydroxy polyesteramides, polysiloxane polyols, polyhydroxy polythioethers, hyperbranched polyols and mixtures thereof.

7. The method according to claim 1, wherein a molar ratio of the at least one alkyl carbamate compound of general formula (I) to the at least one compound having at least one hydroxy group of 1.5 to 5 is used.

8. The method according to claim 1, wherein the at least one tin containing catalyst is selected from the group consisting of dialkyltin alkanoates, dialkyl tin oxides and mixtures thereof.

9. The method according to claim 1, wherein the at least one tin containing catalyst is used in a total amount of 0.01 to 1 wt. % based on the total amount of the at least one alkyl carbamate compound of general formula (I) and the at least one compound having at least one hydroxy group.

10. The method according to claim 1, wherein the reaction is carried out at a temperature in the range of from 110° C. to 140° C.

11. The method according to claim 1, wherein at least 75% of the hydroxy groups present in the at least one compound comprising at least one hydroxy group are replaced with carbamate groups.

12. A carbamate-functional material prepared by the method according to claim 1.

13. A coating composition comprising at least one carbamate-functional material according to claim 12.

14. A coating composition according to claim 13, wherein the coating composition is a clearcoat composition or a tinted clearcoat composition.

15. A method of coating a substrate, comprising applying the coating composition according to claim 13 at least partly to the substrate and curing said applied coating composition.

16. The method according to claim 1, wherein R¹ in general formula (I) is selected from the group consisting of aliphatic tertiary C₄ to C₁₀ alkyl residues.

17. The method according to claim 1, wherein R¹ in general formula (I) is selected from the group consisting of aliphatic tertiary C₄ to C₆ alkyl residues.

18. The method according to claim 1, wherein R¹ in general formula (I) is selected from the group consisting of aliphatic tertiary C₄ residues.

19. The method according to claim 1, wherein the at least one compound having at least one hydroxy group has an average OH-functionality of 1.8 to 8.

20. The method according to any of the preceding claims claim 1, wherein the at least one compound having at least one hydroxy group has an average OH-functionality of 1.8 to 6.
